(19) United States
(12) Patent Application Publication
(10) Pub. No.: US 2025/0281147 A1
(43) Pub. Date: Sep. 11, 2025

(54) BLADDER VOLUME MEASURING DEVICE

(71) Applicant: EDGECARE INC., Seoul (KR)

(72) Inventors: Mann Sik CHUNG, Seoul (KR); Il Seob SONG, Goyang-si (KR); Jung Jun KIM, Seoul (KR)

(21) Appl. No.: 19/051,334

(22) Filed: Feb. 12, 2025

(30) Foreign Application Priority Data
Mar. 7, 2024 (KR) 10-2024-0032582

(51) Int. Cl. A61B 8/08; A61B 8/00; G06T 7/62

(52) U.S. Cl. CPC A61B 8/08; A61B 8/4483; A61B 8/5223; A61B 8/54; G06T 7/62; G06T 2207/10132; G06T 2207/30004

(57) ABSTRACT

A bladder volume measuring device according to an embodiment of the present disclosure may include an ultrasound probe and a calculation unit. The ultrasound probe may transmit an ultrasound signal to an inside of a human body and receive an ultrasound signal reflected from the inside of the human body. The calculation unit may calculate a volume of a bladder included in an ultrasound image generated based on the reception ultrasound signal according to an area of the bladder.

In the bladder volume measuring device according to the present disclosure, the amount of urine contained in a bladder may be more accurately measured by detecting the maximum bladder area among the areas of the bladder included in ultrasound images generated at every scan interval, while an ultrasound probe scans the inside of the human body in the first direction, and calculating the volume of the bladder.

### CROSS-REFERENCE TO RELATED APPLICATION(S)

This application claims benefit of priority to Korean Patent Application No. 10-2024-0032582 filed on Mar. 7, 2024 in the Korean Intellectual Property Office, the disclosure of which is incorporated herein by reference in its entirety.

### BACKGROUND

### 1. Field

The present disclosure relates to a bladder volume measuring device.

### 2. Description of Related Art

When urine is sufficiently stored in the bladder, a normal person may feel that the bladder is filled with enough urine to urinate and discharge it to the outside. However, the number of patients who have difficulty in urinating because they cannot feel that the bladder is sufficiently filled with urine has been increased recently. Various studies have been conducted to solve this problem.

### RELATED ART DOCUMENT

### Patent Document

Korean Application Publication No. 10-2007-0105097 (published on Oct. 30, 2007)

### SUMMARY

An aspect of the present disclosure may provide a bladder volume measuring device capable of more accurately measuring the amount of urine contained in a bladder by detecting the maximum bladder area among the areas of the bladder included in ultrasound images generated at every scan interval, while an ultrasound probe scans the inside of a human body in a first direction, and calculating the volume of the bladder.

According to an aspect of the present disclosure, a bladder volume measuring device includes: an ultrasound probe and a calculation unit. The ultrasound probe may transmit an ultrasound signal to an inside of a human body and receive an ultrasound signal reflected from the inside of the human body. The calculation unit may calculate a volume of a bladder included in an ultrasound image generated based on the reception ultrasound signal according to an area of the bladder.

The calculation unit may include an image providing unit and a scan adjusting unit. The image providing unit may provide the ultrasound images at every scan interval, while the ultrasound probe scans the inside of the human body in a first direction. The scan adjusting unit may adjust the scan interval according to the area of the bladder included in the ultrasound images generated at every scan interval.

The scan adjusting unit may reduce the scan interval as the area of the bladder generated at every scan interval increases.

The calculation unit may further include a maximum unit and a volume unit. The maximum unit may calculate a largest maximum area among the areas of the bladder included in the ultrasound images generated at every scan interval. The volume unit may calculate the volume of the bladder according to the maximum area.

An ultrasound array included in the ultrasound probe may include a first ultrasound array and a second ultrasound array, and the second ultrasound array may be disposed perpendicular to the first ultrasound array at one end of the first ultrasound array.

The calculation unit may further include a directional image providing unit. When the ultrasound probe is placed at a first point of the human body, the directional image providing unit may generate first directional ultrasound images based on a first directional reception ultrasound signal received using the first ultrasound array and generate second directional ultrasound images based on a second directional reception ultrasound signal received using the second ultrasound array.

The calculation unit may further include a first area scoring unit and a second area scoring unit. The first area scoring unit may score areas of the bladder included in the first directional ultrasound images and provide a first area score; and the second area scoring unit may score areas of the bladder included in the second directional ultrasound images and provide a second area score.

The calculation unit may further include a ranking determination unit and a comparison unit. The ranking determination unit may determine priorities of the first area score and the second area score. The comparison unit may compare a higher area score with a higher priority among the first area score and the second area score with a predetermined reference score and provide a comparison result.

The calculation unit may further include a weighting unit, a weighting scoring unit, and a summing unit. The weighting unit may provide weight values applied to a lower area score with a lower priority and the higher area score among the first area score and the second area score. The weighting scoring unit may apply the weight values to the higher area score and the lower area score to provide weighted scores. The summing unit may sum the weighted scores to provide a sum score.

The calculation unit may further include a result unit and a volume calculation unit. The result unit may compare the sum score with a predetermined reference sum score to provide a sum result. The volume calculation unit may calculate the volume of the bladder according to the sum result.

### BRIEF DESCRIPTION OF DRAWINGS

FIG. 1 is a diagram illustrating a bladder volume measuring device according to embodiments of the present disclosure.

FIG. 2 is a diagram illustrating an image providing unit and a scan adjusting unit included in the bladder volume measuring device of FIG. 1.

FIGS. 3 to 5 are diagrams illustrating the image providing unit and the scan adjusting unit of FIG. 2.

FIG. 6 is a diagram illustrating a maximum unit and a volume unit included in the bladder volume measuring device of FIG. 1.

FIG. 7 is a diagram illustrating an example of an ultrasound probe included in the bladder volume measuring device of FIG. 1.

FIGS. 8 to 10 are diagrams illustrating an example of operation of the bladder volume measuring device of FIG. 1.

FIG. 11 is a diagram illustrating a ranking determination unit and a comparison unit included in the bladder volume measuring device of FIG. 1.

FIG. 12 is a diagram illustrating another example of operation of the bladder volume measuring device of FIG. 1.

### DETAILED DESCRIPTION

In the specification, in adding reference numerals to components throughout the drawings, it is to be noted that like reference numerals designate like components even though components are shown in different drawings.

Meanwhile, meanings of the terms described in this specification should be understood as follows.

Terms of singular forms used herein are intended to include their plural forms unless explicitly indicated otherwise, and a scope of the present disclosure is not limited by the terms used herein.

It is to be understood that a term “include” or “have” does not preclude the presence or addition of one or more other features, numerals, operations, components, parts or combinations thereof, which is mentioned in the specification.

Hereinafter, embodiments of the present disclosure are described in detail with reference to the accompanying drawings.

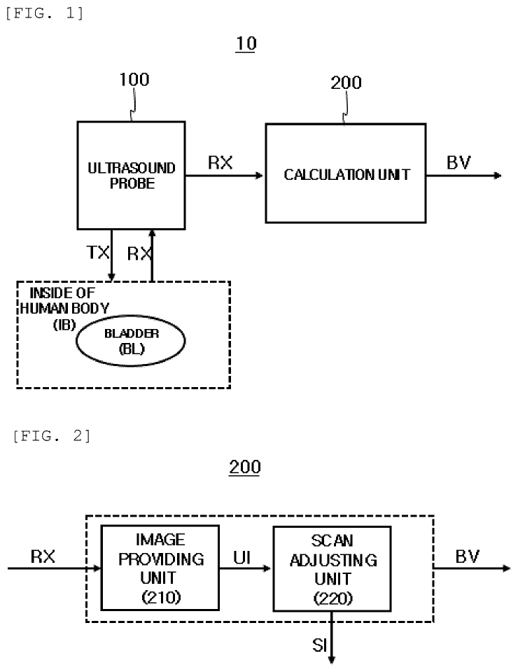

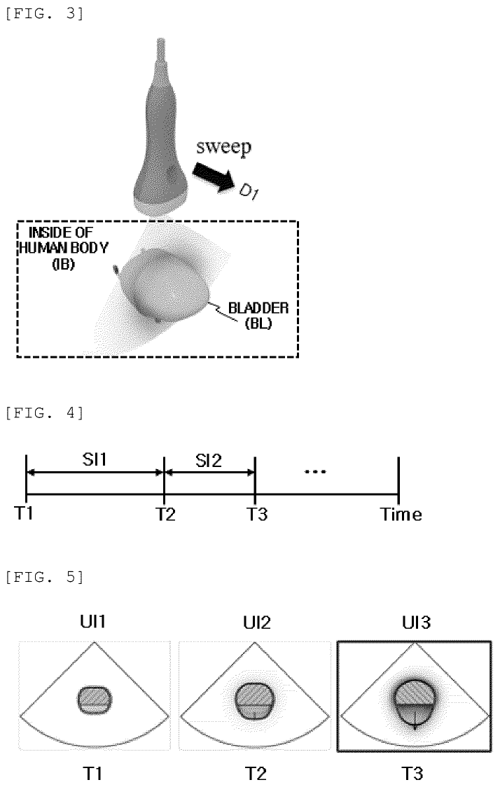

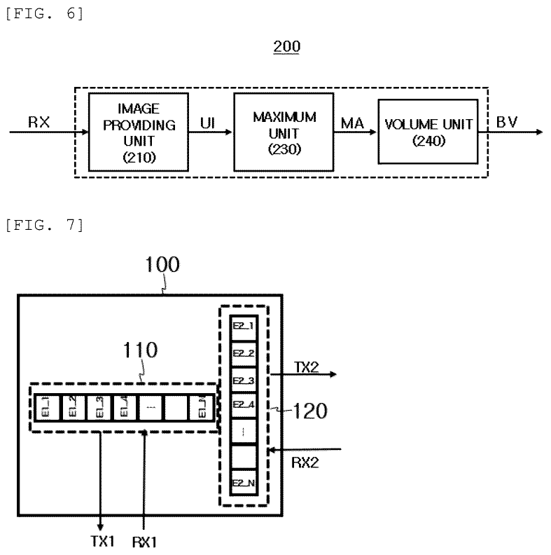

FIG. 1 is a diagram illustrating a bladder volume measuring device according to embodiments of the present disclosure, FIG. 2 is a diagram illustrating an image providing unit and a scan adjusting unit included in the bladder volume measuring device of FIG. 1, FIGS. 3 to 5 are diagrams illustrating the image providing unit and the scan adjusting unit of FIG. 2, and FIG. 6 is a diagram illustrating a maximum unit and a volume unit included in the bladder volume measuring device of FIG. 1.

Referring to FIGS. 1 to 6, a bladder volume measuring device 10 according to an embodiment of the present disclosure may include an ultrasound probe 100 and a calculation unit 200. The ultrasound probe 100 may transmit a transmission ultrasound signal TX to the inside of a human body IB and receive a reception ultrasound signal RX reflected from the inside of the human body IB. For example, by using the ultrasound probe 100, the transmission ultrasound signal TX is transmitted to a region in which a bladder BL of the human body is located, and the reception ultrasound signal RX reflected from the human body is received by the ultrasound probe 100, and then the reception ultrasound signal RX is signal-processed to implement an ultrasound image UI including the bladder BL of the human body.

The calculation unit 200 may calculate a volume BV of the bladder BL according to an area of the bladder BL included in the ultrasound image UI generated based on the reception ultrasound signal RX. In an embodiment, the calculation unit 200 may include an image providing unit 210 and a scan adjusting unit 220. The image providing unit 210 may provide ultrasound images UI at every scan interval SI while the ultrasound probe 100 scans the inside of the human body IB in a first direction D1. For example, a plurality of times may include a first time T1, a second time T2, and a third time T3. A time interval from the first time T1 to the second time T2 may be a first scan interval SI1, and a time interval from the second time T2 to the third time T3 may be a second scan interval. The first scan interval SI1 and the second scan interval SI2 may be the same or different.

In addition, for example, a region in which the bladder BL is placed in the human body IB may be scanned in the first direction D1 using the ultrasound probe 100. A first ultrasound image UI1 may be implemented based on a reception ultrasound signal RX that scans the region in which the bladder BL is placed at the first time T1, and a second ultrasound image UI2 may be implemented based on a reception ultrasound signal RX that scans the region in which the bladder BL is placed at the second time T2 after the first scan interval SI1 from the first time T1. In addition, a third ultrasound image UI3 may be implemented based on a reception ultrasound signal RX that scans the region in which the bladder BL is placed at the third time T3 after the second scan interval SI2 from the second time T2.

The scan adjusting unit 220 may adjust the scan interval SI according to the area of the bladder BL included in the ultrasound images UI generated at every scan interval SI. In an embodiment, the scan adjusting unit 220 may decrease the scan interval SI as the area of the bladder BL generated at every scan interval SI increases. For example, the area of the bladder BL included in the first ultrasound image UI1 captured at the first time T1 may be the smallest, and the area of the bladder BL included in the second ultrasound image UI2 captured at the second time T2 may be the second smallest. In addition, the area of the bladder BL included in the third ultrasound image UI3 captured at the third time T3 may be the largest. Here, the area of the bladder BL may increase as time passes from the first time T1 to the third time T3. In this case, the scan adjusting unit 220 may configure the second scan interval SI2 to be narrower than the first scan interval SI1 in order to more effectively detect the maximum bladder area.

In an embodiment, the calculation unit 200 may further include a maximum unit 230 and a volume unit 240. The maximum unit 230 may calculate the largest maximum area MA among the areas of the bladder BL included in the ultrasound images UIs generated at every scan interval SI. For example, the largest maximum area MA among the areas of the bladder BL included in the first ultrasound image UI1 to the third ultrasound image UI3 may be the area of the bladder BL included in the third ultrasound image UI3.

The volume unit 240 may calculate the volume BV of the bladder BL according to the maximum area MA. For example, when the bladder BL included in the third ultrasound image UI3 is determined as the maximum area MA, the volume unit 240 may calculate the volume BV of the bladder BL according to various methods including a method of measuring the volume BV of the bladder BL using the major and minor axes of the bladder BL included in the third ultrasound image UI3.

In the bladder volume measuring device 10 according to the present disclosure, the volume BV of the bladder BL is calculated by detecting the maximum bladder area among the areas of the bladder BL included in the ultrasound images UI generated at every scan interval SI, while the ultrasound probe 100 scans the inside of the human body IB in the first direction D1, thereby accurately measuring the amount of urine included in the bladder BL.

In addition, in the present disclosure, a unidirectional method for calculating the volume BV of the bladder BL is described, but the present disclosure is not limited thereto, and a case in which the volume is maximum by combining ultrasound images, volumes, or major and minor axes of targets, or other parameters in various directions may be adopted.

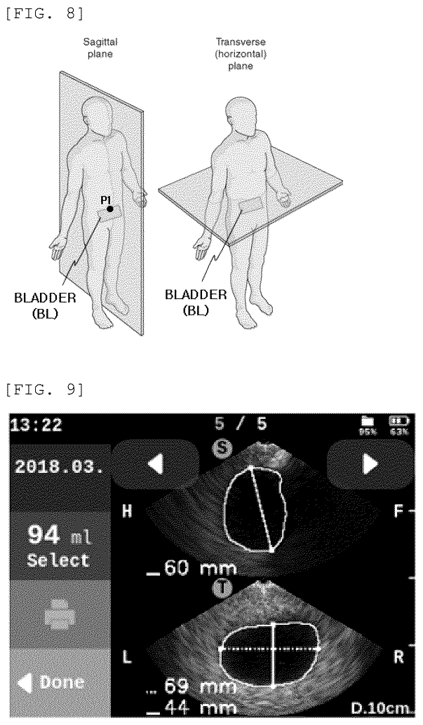

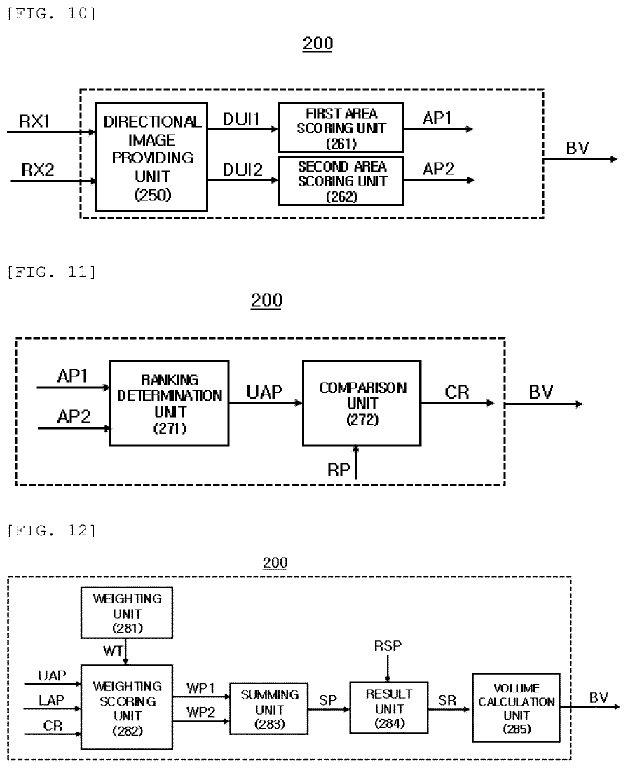

FIG. 7 is a diagram illustrating an example of an ultrasound probe included in the bladder volume measuring device of FIG. 1, and FIGS. 8 to 10 are diagrams illustrating an example of operation of the bladder volume measuring device of FIG. 1.

Referring to FIGS. 1 to 10, in an embodiment, an ultrasound array included in the ultrasound probe 100 may include a first ultrasound array 110 and a second ultrasound array 120. The second ultrasound array 120 may be disposed perpendicular to the first ultrasound array 110 at one end of the first ultrasound array 110. For example, the first ultrasound array 110 may include a plurality of elements, and the plurality of elements included in the first ultrasound array 110 may include a 1_1 element to a 1_N element. The second ultrasound array 120 vertically disposed at one end of the first ultrasound array 110 may include a plurality of elements, and the plurality of elements included in the second ultrasound array 120 may include a 2_1 element to a 2_N element.

In an embodiment, the calculation unit 200 may further include a directional image providing unit 250. The directional image providing unit 250 may generate first directional ultrasound images DUI1 based on a first directional reception ultrasound signal RX1 received using the first ultrasound array 110 when the ultrasound probe 100 is placed at a first point P1 of the human body. For example, after the first ultrasound array 110 and the second ultrasound array 120 illustrated in FIG. 7 are arranged at the first point P1, a first directional transmission ultrasound signal TX1 may be transmitted to the human body using the first ultrasound array 110, and a first directional reception ultrasound signal RX1 corresponding to an ultrasound signal reflected from the human body may be received. In this case, the directional image providing unit 250 may perform signal processing on the first directional reception ultrasound signal RX1 to generate first directional ultrasound images DUI1. Here, the first directional ultrasound image DUI1 may be an ultrasound image for a transverse plane of the bladder BL, as illustrated in FIG. 8.

Second directional ultrasound images DUI2 may be generated based on a second directional reception ultrasound signal RX2 received using the second ultrasound array 120. For example, after the first ultrasound array 110 and the second ultrasound array 120 illustrated in FIG. 7 are arranged at the first point P1, a second directional transmission ultrasound signal TX2 may be transmitted to the human body using the second ultrasound array 120, and the second directional reception ultrasound signal RX2 corresponding to the ultrasound signal reflected from the human body may be received. In this case, the directional image providing unit 250 may process the second directional reception ultrasound signal RX2 to generate a second directional ultrasound images DUI2. Here, the second directional ultrasound image DUI2 may be an ultrasound image for a sagittal plane of the bladder BL, as illustrated in FIG. 8.

In an embodiment, the calculation unit 200 may further include a first area scoring unit 261 and a second area scoring unit 262. The first area scoring unit 261 may score areas of the bladder BL included in the first directional ultrasound images DUI1 to provide a first area score AP1. The second area scoring unit 262 may score areas of the bladder BL included in the second directional ultrasound images DUI2 to provide a second area score AP2. For example, the first area scoring unit 261 may increase the first area score AP1 as the area of the bladder BL included in the first directional ultrasound images DUI1 increases, and may decrease the first area score AP1 as the area of the bladder BL decreases. In addition, the second area scoring unit 262 may increase the second area score AP2 as the area of the bladder BL included in the second directional ultrasound images DUI2 increases, and may decrease the second area score AP2 as the area of the bladder BL decreases.

FIG. 11 is a diagram illustrating a ranking determination unit and a comparison unit included in the bladder volume measuring device of FIG. 1, and FIG. 12 is a diagram illustrating another example of operation of the bladder volume measuring device of FIG. 1.

Referring to FIGS. 1 to 12, in an embodiment, the calculation unit 200 may further include a ranking determination unit 271 and a comparison unit 272. The ranking determination unit 271 may determine a priority of the first area score AP1 and the second area score AP2. The priority of the first area score AP1 and the second area score AP2 may be determined according to user's settings. An area score with a higher priority may be a higher area score UAP, and an area score with a lower priority may be a lower area score LAP. For example, the first area score AP1 may be 80 points, and the second area score AP2 may be 75 points. The higher area score UAP determined according to the user settings may be the second area score AP2, and the lower area score LAP may be the first area score AP1.

The comparison unit 272 may compare the higher area score UAP with a higher priority among the first area score AP1 and the second area score AP2 with a predetermined reference score RP and provide a comparison result CR. For example, the second area score AP2 corresponding to the higher area score UAP may be 75 points, and the reference score RP may be 77 points. In this case, the comparison unit 272 may provide a comparison result CR that the higher area score UAP is smaller than the reference score RP. When the comparison unit 272 provides the comparison result CR that the higher area score UAP is smaller than the reference score RP, the bladder volume measuring device 10 according to the present disclosure may not calculate the volume BV of the bladder BL.

In an embodiment, the calculation unit 200 may further include a weighting unit 281, a weighting scoring unit 282, and a summing unit 283. The weighting unit 281 may provide a weight value WT applied to the lower area score LAP having a lower priority among the first area score AP1 and the second area score AP2 and the higher area score UAP. For example, the first area score AP1 may be 85 points, and the second area score AP2 may be 80 points. In addition, the reference score RP may be 77 points, and the higher area score UAP may be the second area score AP2. In this case, the second area score AP2, which is the higher area score UAP, may be greater than the reference score RP. When the second area score AP2, which is the higher area score UAP, is greater than the reference score RP, the weighting unit 281, the weighting scoring unit 282, and the summing unit 283 may operate.

For example, the weighting unit 281 may provide a first weight WT1 applied to the higher area score UAP and a second weight WT2 applied to the lower area score LAP, the first weight WT1 may be 2, and the second weight WT2 may be 1.

The weighting scoring unit 282 may provide weighted scores by applying a weight value WT to the higher area score UAP and the lower area score LAP. For example, the second area score AP2 corresponding to the higher area score UAP may be 80 points, and the first area score AP1 corresponding to the lower area score LAP may be 85 points. In this case, a first weighted score WP1 obtained by multiplying the higher area score UAP of 80 points by the first weight WT1 of 2 may be 160 points, and the first weighted score WP1 obtained by multiplying the lower area score LAP of 85 points by the second weight WT2 of 1 may be 85 points.

The summing unit 283 may sum the weighted scores to provide a sum score SP. For example, a plurality of weighted scores may include a first weighted score WP1 and a second weighted score WP2. The first weighted score WP1 may be 160 points, and the second weighted score WP2 may be 85 points. In this case, the sum score SP may be 245 points.

In an embodiment, the calculation unit 200 may further include a result unit 284 and a volume calculation unit 285. The result unit 284 may compare the sum score SP with a predetermined reference sum score RSP and provide a sum result SR. For example, the sum score SP may be 245 points, and the reference sum score RSP may be 230 points. In this case, the result unit 284 may provide a sum result SR in which the sum score SP is greater than the reference sum score RSP.

The volume calculation unit 285 may calculate the volume BV of the bladder BL according to the sum result SR. For example, the volume BV of the bladder BL may be calculated by using the major and minor axes of the bladder BL included in the first directional ultrasound image DUI1 and the second directional ultrasound image DUI2 having the highest sum score SP, among the ultrasound images having the sum result SR in which the sum score SP is greater than the reference sum score RSP.

In the bladder volume measuring device 10 according to the present disclosure, the volume BV of the bladder is calculated by detecting the maximum bladder area among the areas of the bladder BL included in the ultrasound images generated at every scan interval SI while the ultrasound probe 100 scans the inside of the human body IB along the first direction, thereby allowing more accurate measurement of the amount of urine included in the bladder.

As set forth above, the present disclosure as described above may provide the following effects.

In the bladder volume measuring device according to the present disclosure, the amount of urine contained in a bladder may be more accurately measured by detecting the maximum bladder area among the areas of the bladder included in ultrasound images generated at every scan interval, while an ultrasound probe scans the inside of the human body in the first direction, and calculating the volume of the bladder.

In addition, other features and advantages of the present disclosure may be newly identified through the embodiments of the present disclosure.

In addition to the above-mentioned technical tasks of the present disclosure, other features and advantages of the present disclosure may be described below, or may be clearly understood by those skilled in the art to which the present disclosure pertains from such description and explanation.

What is claimed is:

1. A bladder volume measuring device comprising:
an ultrasound probe configured to transmit an ultrasound signal to an inside of a human body and to receive an ultrasound signal reflected from the inside of the human body; and
a calculation unit configured to calculate a volume of a bladder included in an ultrasound image generated based on the reception ultrasound signal according to an area of the bladder.

2. The bladder volume measuring device of claim 1, wherein
the calculation unit includes:
an image providing unit configured to provide the ultrasound images at every scan interval, while the ultrasound probe scans the inside of the human body in a first direction; and
a scan adjusting unit configured to adjust the scan interval according to the area of the bladder included in the ultrasound images generated at every scan interval.

3. The bladder volume measuring device of claim 2, wherein the scan adjusting unit reduces the scan interval as the area of the bladder generated at every scan interval increases.

4. The bladder volume measuring device of claim 3, wherein
the calculation unit further includes:
a maximum unit configured to calculate a largest maximum area among the areas of the bladder included in the ultrasound images generated at every scan interval; and
a volume unit configured to calculate the volume of the bladder according to the maximum area.

5. The bladder volume measuring device of claim 4, wherein an ultrasound array included in the ultrasound probe includes a first ultrasound array and a second ultrasound array, and the second ultrasound array is disposed perpendicular to the first ultrasound array at one end of the first ultrasound array.

6. The bladder volume measuring device of claim 5, wherein the calculation unit further includes a directional image providing unit configured to, when the ultrasound probe is placed at a first point of the human body, generate first directional ultrasound images based on a first directional reception ultrasound signal received using the first ultrasound array and to generate second directional ultrasound images based on a second directional reception ultrasound signal received using the second ultrasound array.

7. The bladder volume measuring device of claim 6, wherein
the calculation unit further includes:
a first area scoring unit configured to score areas of the bladder included in the first directional ultrasound images and to provide a first area score; and
a second area scoring unit configured to score areas of the bladder included in the second directional ultrasound images and to provide a second area score.

8. The bladder volume measuring device of claim 7, wherein
the calculation unit further includes:
a ranking determination unit configured to determine priorities of the first area score and the second area score; and
a comparison unit configured to compare a higher area score with a higher priority among the first area score and the second area score with a predetermined reference score and to provide a comparison result.

9. The bladder volume measuring device of claim 8, wherein
the calculation unit further includes:
a weighting unit configured to provide weight values applied to a lower area score with a lower priority and the higher area score among the first area score and the second area score;
a weighting scoring unit configured to apply the weight values to the higher area score and the lower area score to provide weighted scores; and
a summing unit configured to sum the weighted scores to provide a sum score.

10. The bladder volume measuring device of claim 9, wherein
the calculation unit further includes:
a result unit configured to compare the sum score with a predetermined reference sum score to provide a sum result; and
a volume calculation unit configured to calculate the volume of the bladder according to the sum result.
